(19) United States
(12) Patent Application Publication
(10) Pub. No.: US 2002/0192268 A1
(43) Pub. Date: Dec. 19, 2002

(54) Substrates utilizing shear responsive micropockets for storage and delivery of substances

(76) Inventors: Ali Abdelaziz Alwattari, Hamilton, OH (US); Rajeev Chhabra, Sharonville, OH (US); Marcia J. Holden, Harrison, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 09/981,345

(22) Filed: Oct. 17, 2001

Related U.S. Application Data
(60) Provisional application No. 60/299,250, filed on Jun. 19, 2001.

(51) Int. Cl. A61K 9/70; B32B 27/12

(52) U.S. Cl. 424/443; 442/123

(57) ABSTRACT

The present invention relates to substrates such as wipe articles for use in combination with substances, such as ingredients and formulations, for delivery to the human body or for use on inanimate surfaces such as those found in households and/or workplace. The substrate utilizes shear responsive micropockets that store substances, and controllably release said substances when shear forces are applied thereto, while in contact with the target surface. Shear responsive micropockets are fine scale three-dimensional compartments in the surface of the substrate such that applying shear forces along the surface of said substrate while said substrate is in contact with said target surface, said shear forces cause walls of said micropockets to substantially deform, and release said substance, to said target surface.

### FIELD OF INVENTION

The present invention relates to substrates for controlled storage and release of substances. Specifically, the substrate comprises shear responsive micropockets capable of containing and regulating release of substances upon contact with living and synthetic surfaces in articles such as wipes, pads, applicators, sleeves, and disposable attachments to implements, wipers, and so forth.

### BACKGROUND OF THE INVENTION

Substrates or cloths for conditioning, spreading, cleansing, removing, and related functions are well known commercially for both personal and industrial uses. Such products typically comprise paper or non-woven fabric sheets and can comprise substances such as emulsions, suspensions, and in fact any mechanically, theologically, thermally, or wettably transferable substance such as pigments, surfactants, waxes, and medications. Current wipes are typically paper, rayon, cellulose, or polyester substrates. These substrates function by capillary and physio-chemical transport of substances into the fibers and pores of the substrate. Moreover, for applications needing to contain high levels of substances, relatively hygroscopic materials are used. This means that the substance is relatively trapped, requiring large applied forces to release it. Typically, only a small fraction of the entrapped substance is released, leading to early termination of release, limiting the ability to cover large surfaces. By the same token, substrates like polypropylene nonwoven that are not hygroscopic have less substance holding capacity, thus often leak, and release substances too rapidly and uncontrollably. Other substrates such as nonwovens laminated to polymer films prevent leakage of substances; however, said laminates do not provide control or regulation of the rate of release. This problem becomes commercially important when it is necessary to ensure efficient delivery of valuable substances and/or to enable multiple consumer benefits to be achieved simultaneously such as cleaning plus conditioning or moisturizing plus sun protection. Thus, an unmet need is creating a substrate that is not limited to hygroscopic materials yet can store large amounts of a substance with subsequently high release of the substance in the practical form of a wipe article. Applicants have disclosed that higher release, versus hygroscopic substrates, can be achieved using polymeric micropocket films and/or nonwovens. This is because the micropockets are both non-trapping of substances and because the micropockets exhibit shear responsive behavior, and substantially open and release their contents during wiping. Surprisingly, the shear responsive micropockets produce novel benefits including but not limited to the following: more complete and efficient release of stored substance, a high substance storage capacity without the need for hygroscopic materials, and a more regulated and wiping-responsive substance delivery to target surfaces. This innovation of utilizing shear responsive micropockets for substance storage and release is applicable whether the manufacturer pre-loads the substrates with substances or whether the end user loads the substrates, for example, by spraying or spreading a substance of their choice onto the substrate. Accordingly, it is an object of the present invention to provide the above benefits for depositing substances onto living surfaces such as skin, hair, pet fur, teeth or on inanimate surfaces such as hard surfaces, glass, flooring, wood, painted surfaces and so forth. These and other objects, advantages, and novel features will become apparent to those skilled in the art from the following description and appended claims.

### SUMMARY OF THE INVENTION

The present invention relates to a wipe article comprising a substrate and at least one substance, wherein said substrate has a plurality of micropockets. The micropockets have a length, a width, and a depth, wherein the ratio of said depth to said width is at least 1:2. The substance is contained in the micropockets. The substance remains in the micropockets until the substrate is subjected to a shear force while in contact with a target surface. The shear force causes the walls of said micropockets to substantially deform, and release said substance to said target surface.

The present invention further relates to a method of applying at least one substance to a target surface. The method comprises providing a substrate having a plurality of micropockets containing said substance, said micropockets having a width and a depth, wherein the ratio of said depth to said width is at least 1:2. The substrate is placed in contact with said target surface. Shear forces are applied along the surface of said substrate while said substrate is in contact with said target surface, said shear forces causing walls of said micropockets to substantially deform, and release said substance to said target surface.

### BRIEF DESCRIPTION OF THE DRAWINGS

While the specification concludes with claims particularly pointing out and distinctly claiming the present invention, it is believed that the present invention will be better understood from the following description taken in conjunction with the accompanying drawings in which like reference numerals identify like elements.

FIG. 1 is a top plan view of an example embodiment of the present invention, disclosing a piece of substrate having an array of shear responsive micropockets containing a substance.

FIG. 2 is an enlarged partial top plan view of the material of FIG. 1, showing an array of shear responsive micropockets.

FIG. 3 is an elevational sectional view of the material of FIG. 2, taken along section line 3-3, showing the cross-section of shear responsive micropockets filled with a substance.

FIG. 3A is a photograph of an elevational sectional view showing cross-section of an embodiment of shear responsive micropockets in a polyethylene film substrate.

FIG. 4 is a combination of two photographs of a top-plan view of an embodiment of a substrate of the present invention having shear responsive micropockets, with the left photograph showing empty micropockets, and the right photograph showing lotion-filled micropockets in a polyethylene film substrate.

FIGS. 5A-5D are a series of photographs showing elevational sectional view of deformation of walls of an embodiment of a shear responsive micropocket when subjected to shear force along the surface of the substrate during wiping.

FIGS. 6A-6C are a series of photographs showing elevational sectional view of collapsing of walls of a micropocket when subjected to compression force perpendicular to the surface of the substrate.

FIG. 7 is a schematic comparison of release profile of a substrate with shear responsive micropockets relative to a control substrate without any micropockets.

FIG. 8 is an elevational sectional view of an embodiment of the present invention with a combination of two substrates with shear responsive micropockets, wherein openings of micropockets are facing each other.

FIG. 9 is an elevational sectional view of an embodiment of the present invention with a combination of two substrates with shear responsive micropockets, wherein each micropocket of one substrate is nested into a micropocket of the other substrate.

FIG. 10A is an elevational sectional view of an embodiment of the present invention with a combination of two substrates with shear responsive micropockets, wherein a set of micropockets of one substrate is interlocked into a region of the other substrate devoid of micropockets.

FIG. 10B is an elevational sectional view of an embodiment of the present invention with an array of micropockets on both sides of the substrate

FIG. 11 is an elevational sectional view of an embodiment of an array of round bottom shear responsive micropockets in a substrate.

FIG. 12 is an elevational sectional view of an embodiment of an array of bilobal shear responsive micropockets in a substrate.

FIG. 13 is an elevational sectional view of an embodiment of the present invention with a combination of three substrates, wherein two outer substrates have micropockets, and perforations to allow flow of substances to and from the third substrate between the two outer substrates.

FIG. 14 is a plan view photograph of an embodiment of shear responsive micropocket in a polyethylene film substrate, wherein micropockets are protruding out of the surface of the substrate opposite to openings of the micropockets.

FIG. 15 is a top-plan view of a bond pattern for bonding at least two layers of substrates, wherein at least one of the substrates has shear responsive micropockets.

FIG. 16 is a top-plan view of an embodiment of a substrate of FIG. 14 with a substance contained in shear responsive micropockets.

FIG. 17 is an elevational sectional view of an embodiment of a substrate with shear responsive micropockets of two different depths but having the same width.

FIG. 18 is an elevational sectional view of an embodiment of a substrate with shear responsive micropockets of two different widths but having the same depth.

FIGS. 19-27 are plan view illustrations of other preferred embodiments of shapes and arrangements of shear responsive micropockets on substrates comprising the present invention.

### DETAILED DESCRIPTION OF THE INVENTION

As Used Herein:

“Substrate” refers to a solid state material made of films, nonwovens, porous membranes, porous films, or a thin coating on another object or durable item.

“Substance” refers to a composition—either single material or formulation or reactive mixture—that is contained, stored, or otherwise immobilized in the substrate. The composition can be a liquid, gas, powder, or any manifestation of these states of matter such as self-foaming gel, semi-solid, paste, stick, gum, emulsion, water, suspension, dispersion, aqueous latex, particle, microsphere, and the like. The substance is capable of being transferred from a containment vehicle like a substrate onto a surface.

“Film” refers to a polymer film including flat nonporous films, and porous films such as microporous, nanoporous, closed or open celled, breathable films, or apertured films.

“Nonwoven” refers to a structure of individual fibers or threads, which are interlaid to form a piece of material that is in the form of a substrate. These can be formed by a variety of processes, including but not limited to melt blowing, spun bonding, bonded carded, thermally induced phase separation following by stretching and ringing, and hydroentangling.

“Wiping” refers to any shearing action that the substrate undergoes while in contact with a target surface. This includes hand or body motion, substrate-implement motion over a surface, or any perturbation of the substrate via energy sources such as ultrasound, mechanical vibration, electromagnetism, and so forth.

“Loading” refers to applying substances to the substrate in preparation for substance delivery to target surfaces. Methods of loading include immersion, squeegeeing, spraying, dot matrix deposition, slot coating, printing, filling, dipping, and individual ingredient imprinting on selected regions of the substrate laterally or at different depths of the substrate.

“Percent release” refers to how much of the loaded material comes out of the substrate after wiping. This is calculated as the difference between the amount of substance originally loaded onto the substrate and the amount of substance removed from the substrate after consumption divided by the amount of substance originally loaded onto the substrate; this quantity is then multiplied by one hundred to get percent substance released.

“Responsive” refers to deformation of substrate micropockets during the shearing that accompanies wiping action. While not being limited by theory, it is believed the micropockets are substantially closed at rest, then as a result of the shearing forces during motion and contact with the target surface, the micropockets substantially open, causing flow of the substance to the target surface.

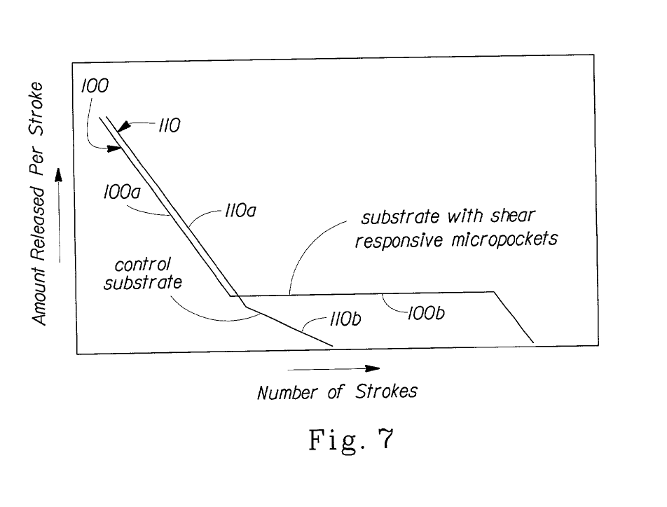

“Sustained release” refers to an increased percent release. This is illustrated in FIG. 7, which demonstrates how a micropocket film laminated to a nonwoven has more sustained release at the same mass loading relative to a control of flat film laminated to a nonwoven.

“Sequential release” refers to consecutive stages of release of one or more substances. For example, the same substance can be located at two layers of the substrate and hence one portion releases before the other portion of substance. Alternatively, one substance can be stored in one location and a completely independent substance can be stored in another location of the substrate. For example, one layer of film with micropockets is laminated to another layer of porous nonwoven with micropockets. This is particularly valuable in delivery of substances that perform separate and distinct functions such as conditioning and cleaning; for example, solid soap and liquid skin conditioner. This permits individual ingredients to be combined without the need for special formulations and emulsifiers. By different location, this can mean at two locations of the same layer of micropockets or at two different layers or at two different times.

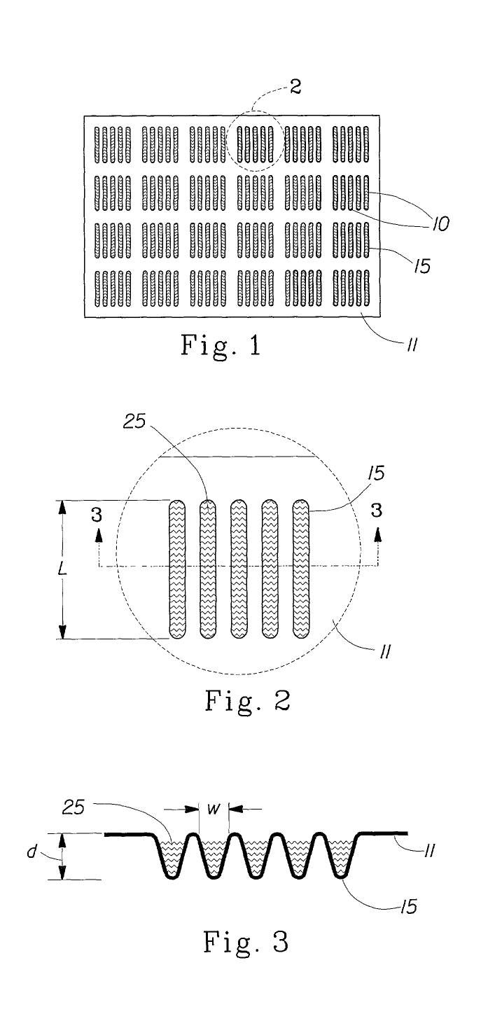

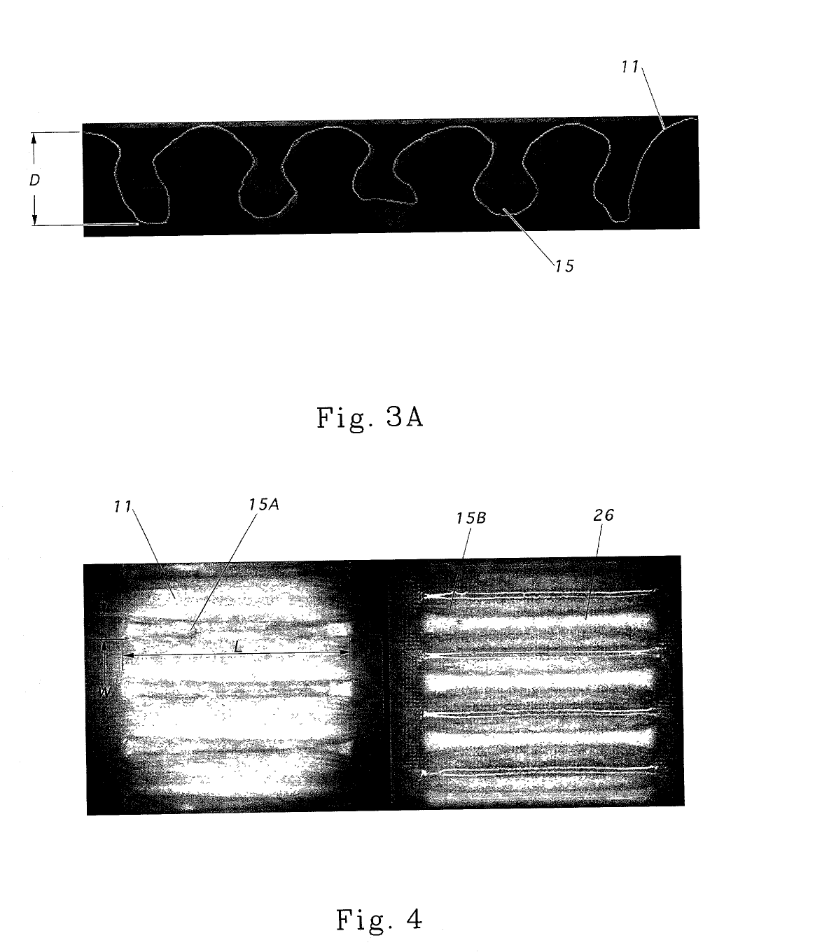

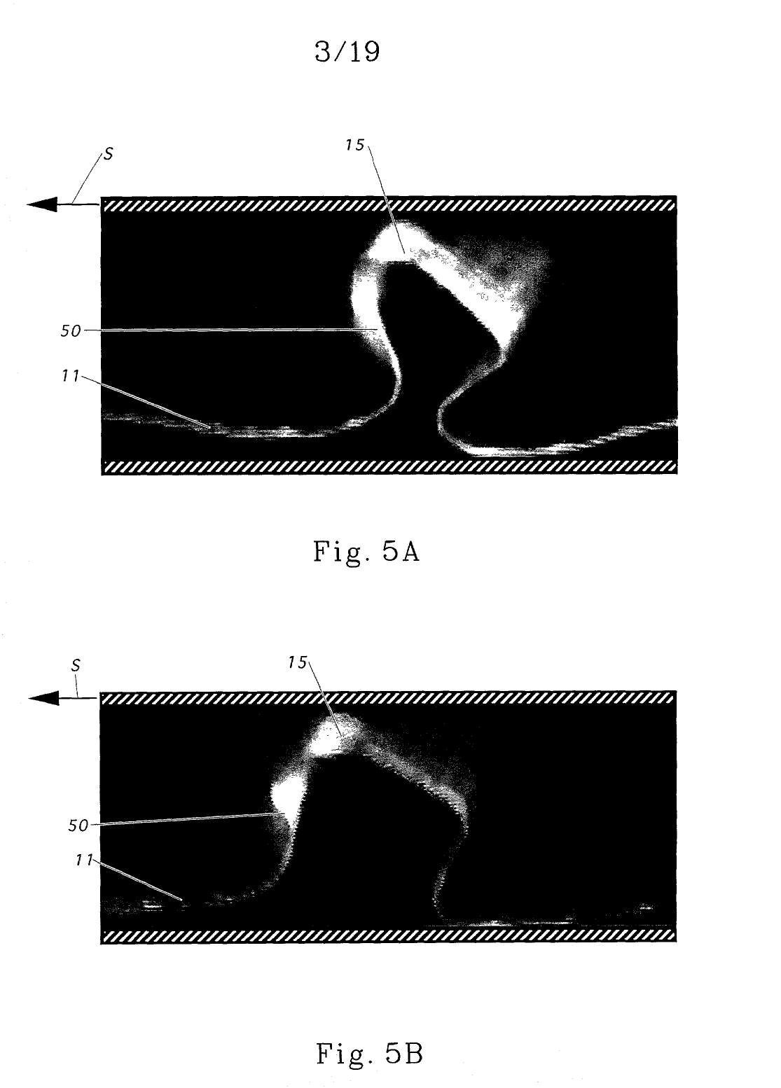

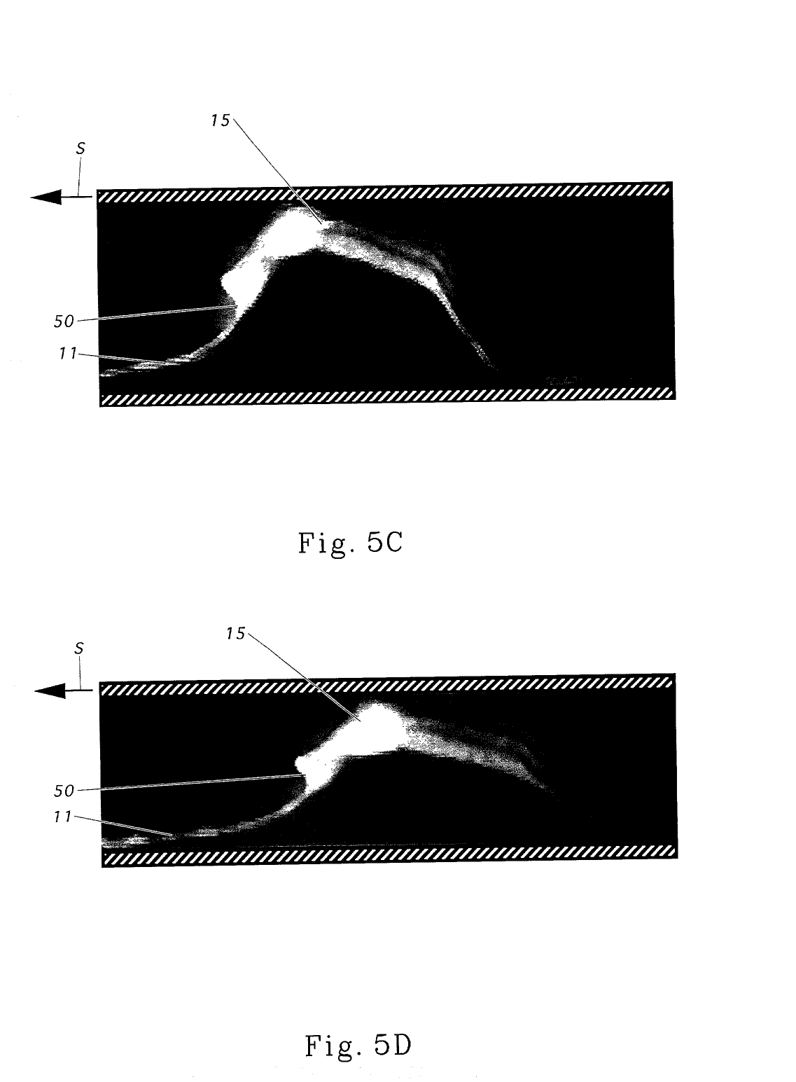

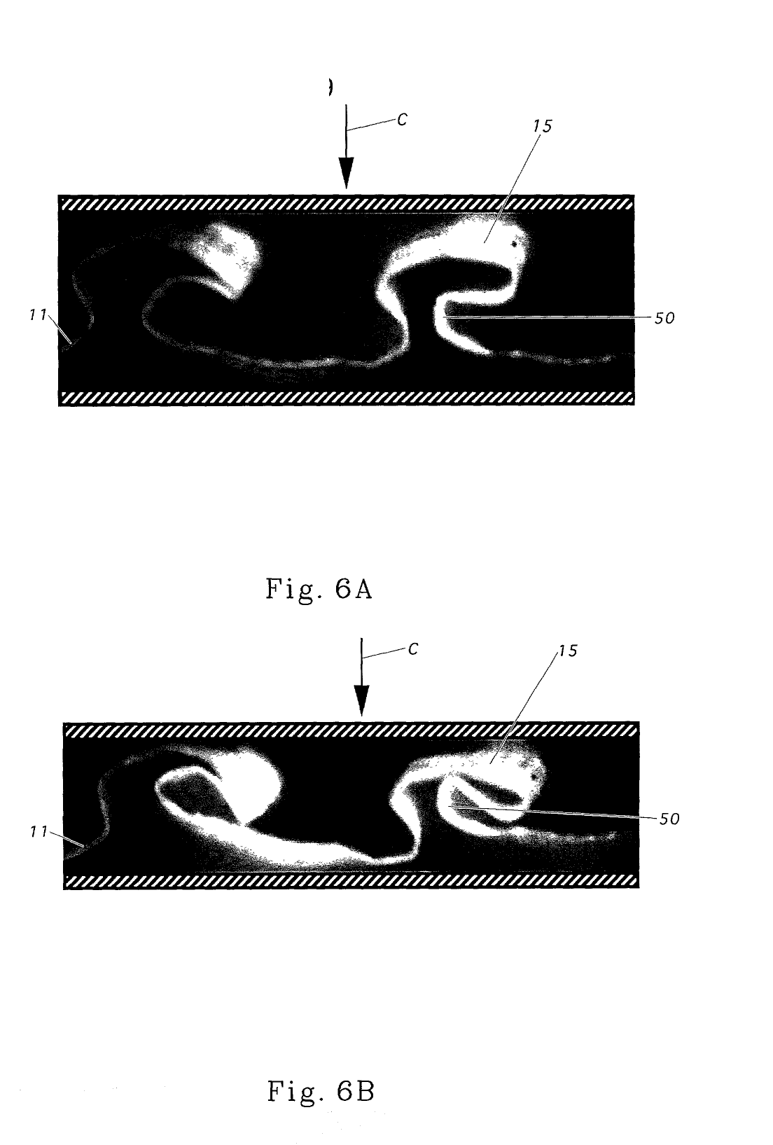

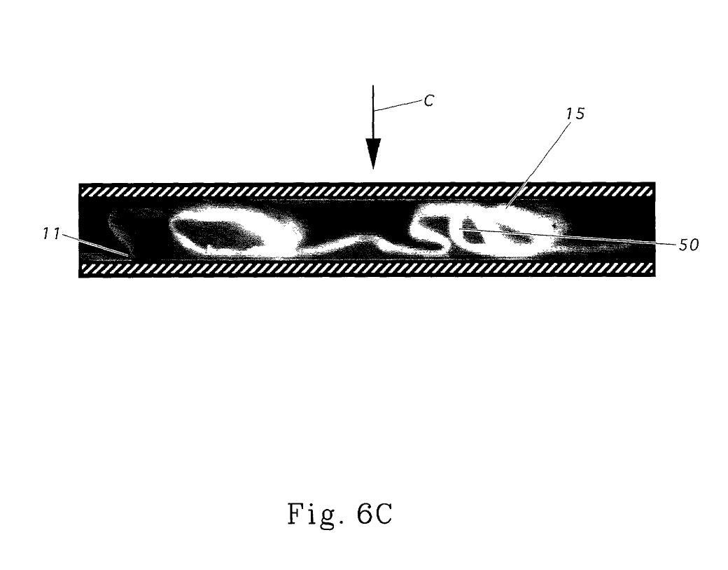

FIGS. 1-4 illustrate an example embodiment of the present invention of shear responsive micropockets on a substrate. A shear responsive micropocket containing substances is a fine scale three-dimensional compartment in the surface of the substrate 11, typically with the compartment depth D having the same or a greater size scale than the compartment width W (as shown in FIG. 3), and with the compartment walls equally or more flexible than the substrate regions that are devoid of micropockets. FIG. 3 illustrates an example embodiment of a cross-section of a micropocket 15 in the form of a trapezoid, and containing a substance 25 in a substrate 11. FIG. 3A illustrates a photograph of a similar embodiment of a cross-section of a micropocket 15 in a polyethylene film 11. FIG. 4 shows a photograph of another embodiment of an empty micropocket 15A and a micropocket 15B filled with lotion 26 in a polyethylene film 11. The size scale of the micropocket is substantially smaller than that of a typical substrate article such as a cloth or a wet wipe wherein micropockets exist. When a plurality of these micropockets, as exemplified by 10 in FIGS. 1-4, coexist in an array in a substrate 11, the walls 50 of an individual micropocket 15, as illustrated in FIGS. 5A-5D, substantially deform in a direct response to a shearing force S that occurs during wiping of target surfaces. As shown in FIGS. 5A-5D, the shear force S acts along a surface of the polyethylene film substrate 11. The deformation of micropockets causes the contained substance to flow out of the deformed micropockets. This use, for example, includes but is not limited to applying the substrate to a surface for purposes of cleaning, conditioning, or otherwise delivering contained substance to the surface. Referring to FIGS. 6A-6C, if compression force C is acting on the substrate 11 with micropocket 15, the walls 50 of the micropocket collapse and close the micropocket preventing the release of substance in micropocket. The shear responsive micropocket dimensions can be varied depending on the viscosity of the substance to be stored and released, and the wettability of the substrate by the substance. For example, to store and release a low viscosity substance such as cleaning fluid or water from a polyethylene micropocket film, useful micropocket dimensions, as illustrated in FIGS. 1-4, are: width W from about 0.4 to about 2 millimeters, depth D from about 0.3 to about 1.3 millimeters, and length L from about 1 to about 15 millimeters. To store and release a more viscous substance such as cream or lotion, useful micropocket dimensions are: width W from about 0.5 to about 2 millimeters, depth D from about 0.8 to about 1.3 millimeters, and length L from about 1 to about 15 millimeters.

Substrates with shear responsive micropockets and subsequent combination of said substrates with substances results in articles that are physically suited to the natural anatomical motion of hand wiping which occurs in the use of wipe articles across a broad range of tasks such as removing, cleaning, polishing, conditioning, moisturizing, washing, depositing, coating, spreading and so forth. An example of a suitable substrate material for use in the present invention is described in U.S. Pat. No. 5,968,029 Chappell et al., incorporated herein by reference. The surprising finding of the present invention is that shear responsive micropockets lead to control of both substance storage and release.

A need also exists to store and release a range of substance rheologies from solid substances to flowable and low viscosity substances. Substrates comprising shear responsive micropockets fulfill this need by allowing a wide range of rheologies to be both stored and controllably released. The micropockets act as compartments that are adaptable in size, shape, and pattern to match the theological and surface properties of the substance to be delivered. While primarily triggered by shearing during wiping, the micropocket substrates of the present invention can additionally be triggered by abrasion, heating to cause flow, and wettable dispersion or loosening of ingredients followed by releasing from the article.

The present invention can comprise, consist of, or consist essentially of the essential elements of the invention described herein, as well any of the additional or optional ingredients, and components described herein. The present invention relates to substrate articles which provide improved storage and release performance leading to better sustainment and completeness of release, better value, ease of use, and diversity of substances deliverable. The essential and optional components used to prepare the substrates of the present invention, as well as the process for preparing and using them, are described in detail as follows.

### Essential Components

Substrates

The substrate material comprises shear responsive micropockets combined with fibrous substrates including nonwovens, wovens, knits and/or film substrates including films, membranes, sheet, apertured films, foams, and elastic substrates, or combinations thereof. The substrates of the present invention contain substances and release them in response to consumer use.

Shear Responsive Micropockets

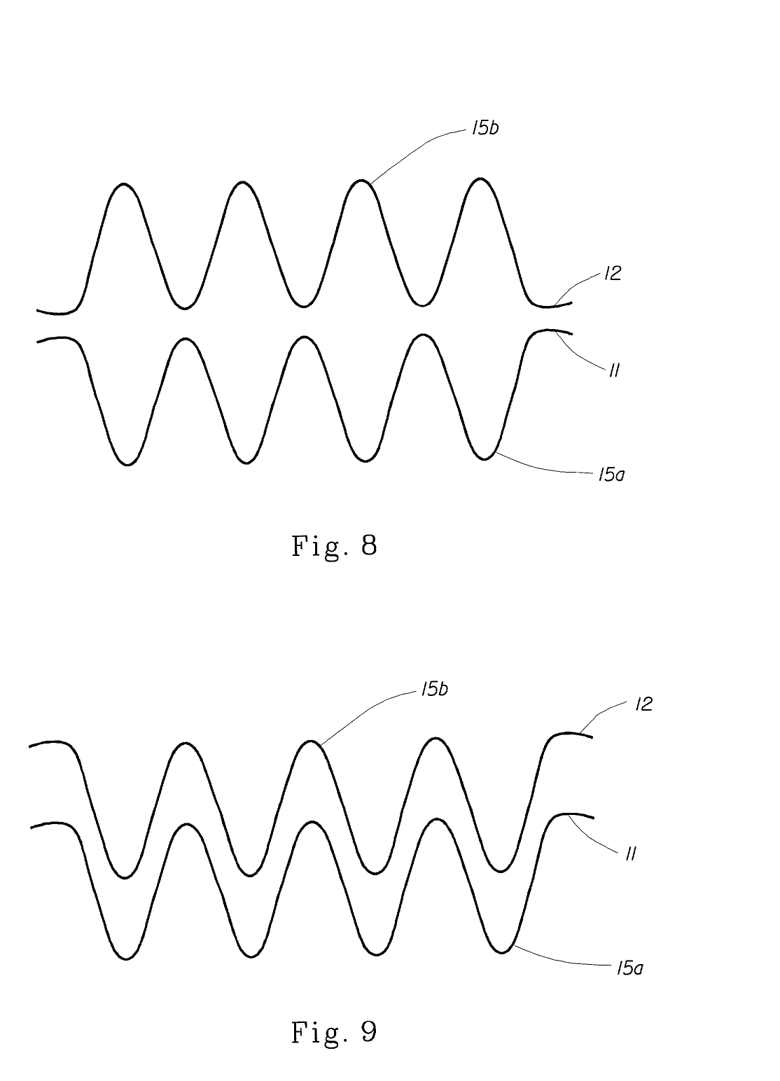

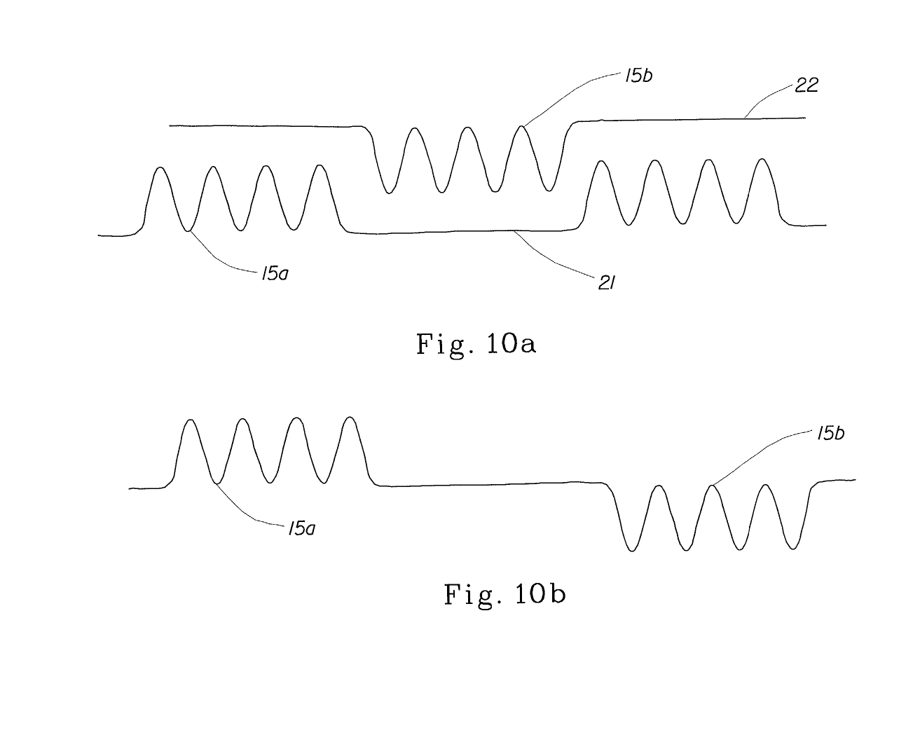

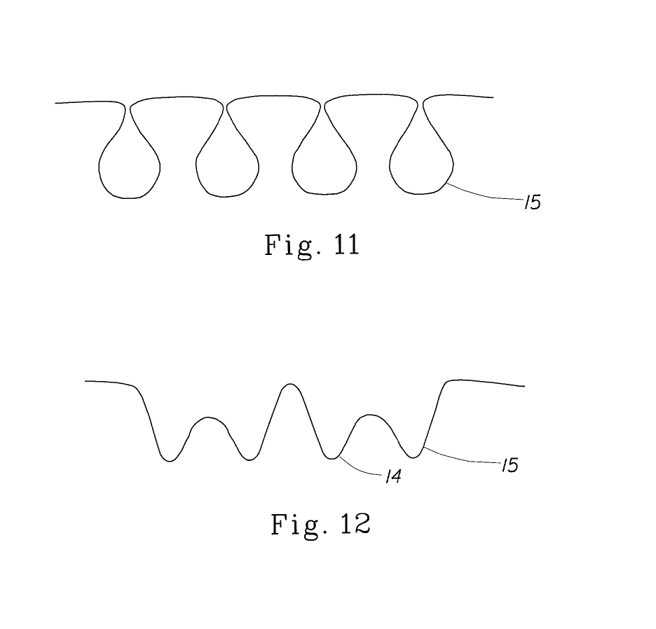

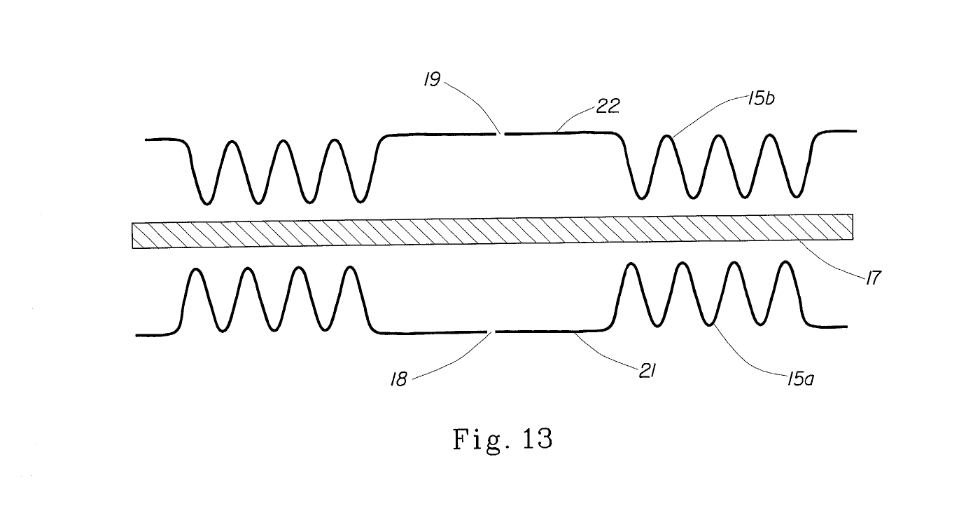

Shear responsive micropockets are fine scale three-dimensional compartments in the surface of the substrate, typically with the compartment depth having the same or a greater size scale than the compartment width, and with the compartment walls equally or more flexible than the substrate regions that are devoid of micropockets. The form of individual micropockets is also variable, for example, nonporous and porous micropockets; back to back combination of micropocketed substrate layers 11 and 12, as illustrated in FIG. 8; micropockets on one side of a substrate layer; nested micropockets 15A and 15B as illustrated in FIG. 9; interlocking micropocket sets 15A and 15B, as illustrated in FIG. 10A; micropockets 15A and 15B on both sides of any given substrate layer, as illustrated in FIG. 10B; rounded and multi-lobal micropockets 15, as illustrated in FIGS. 11 and 12 respectively; and so on. Apart from the form of micropockets, a combination of micropocketed substrates with non-micropocketed substrates is possible; for example, FIG. 13 illustrates an embodiment of a combination of micropocketed substrate layers 21 and 22 with perforations 18 and 19, and another optional substrate layer 17 in between the micropocketed layers.

The approach of the present invention to achieve substance storage and release is to utilize substrates with micropockets formed on them such that under static conditions, the substance loaded into the substrate is stored, and then under shear, the substance is released from the micropockets. As an illustration of this point, a polyethylene film in the form of a 150 mm by 200 mm sheet can contain 2000 such micropockets in a uniform or random array; for example, an embodiment of such micropocket array is shown in FIG. 1. When this micropocket film sheet is bonded to a nonwoven, for example, a wipe substrate is produced that can be loaded, and controllably releases substances in a uniform and sustained way. When a control experiment is conducted with a wipe made with a flat film of the same substrate material devoid of micropockets and bonded to a nonwoven, a poor control of release occurs, such that the substance releases over a short duration and non-uniformly. In the case of fibrous substrates, such as nonwovens with micropockets, there are “porous micropockets” and regions of small and large pores created throughout the nonwoven substrate.

Fibrous Substrates

The substrate material may also be comprised of a synthetic woven, synthetic knit, knit and durable fabrics, woven fabrics, nonwoven, fibrous or particulate materials, or laminates and combinations thereof. The nonwovens may be made by, but not limited to, any of the following methods: spunlace, spun bonding, melt blowing, carding, hydroaperturing, hydroentangling or hydraulically entangling, air through bonding, calendar bonding, or combinations thereof. Nonwoven substrates may be comprised of a variety of materials both natural and synthetic. By natural is meant that the materials are derived from plants, animals, insects, or byproducts of plants, animals, and insects. By synthetic is meant that the materials are obtained primarily from various man-made materials or from natural materials that have been further altered. The conventional base starting material is usually a fibrous substrate comprising any of the common synthetic or natural textile-length fibers, or mixtures thereof. Non-limiting examples of natural materials useful in the present invention are silk fibers, keratin fibers, and cellulosic fibers. Non limiting examples of keratin fibers include those selected from a group consisting of wool fibers, camel hair fibers, and the like. Non-limiting examples of cellulosic fibers include those selected from the group consisting of wood pulp fibers, cotton fibers, hemp fibers, jute fibers, flax fibers, and mixtures thereof. Non limiting examples of synthetic materials useful in the present invention include those selected from a group consisting of acetate fibers, acrylic fibers, cellulose ester fibers, polyamide fibers, polyester fibers, polyolefin fibers, polyvinyl alcohol fibers, rayon fibers, polyurethane foam, and mixtures thereof. Examples of some of these synthetic materials include acrylics such as acrilan, creslan, and the acrylonitrile-based fiber, orlon; cellulose ester fibers such as cellulose acetate, arnel, and acele; polyamides such as nylons (e.g., nylon 6, nylon 66, nylon 610, and the like); polyesters such as fortrel, kodel, and the polyethylene terephthalate fiber, dacron; polyolefins such as polypropylene, polyethylene; polyvinyl acetate fibers; polyurethane foams and mixtures thereof. These and other suitable fibers and the nonwoven materials prepared there from are generally described in Riedel, “Nonwoven Bonding Methods and Materials,” Nonwoven World (1987); and The Encyclopedia Americana, vol. 11, pp. 147-153, and vol. 26, pp. 566-581 (1984) which are all incorporated by reference herein in their entirety. Nonwoven substrates made from natural materials consist of substrates or sheets most commonly formed on a fine wire screen from a liquid suspension of the fibers. See C. A. Hampel et al., The Encyclopedia of Chemistry, third edition, 1973, pp. 793-795 (1973); The Encyclopedia Americana, vol. 21, pp. 376-383 (1984); and G. A. Smook, Handbook of Pulp and Paper Technologies, Technical Association for the Pulp and Paper Industry (1986); which are incorporated by reference herein in their entirety. Substrates made from natural materials useful in the present invention can be obtained from a wide variety of commercial sources. Non limiting examples of suitable commercially available paper layers useful herein include AirtexR, Polypropylene/Rayon from Suominen, an embossed airlaid cellulosic layer available from James River, Green Bay, Wis.; and WalkisoftR, an embossed airlaid cellulosic layer available from Walkisoft U.S.A., Mount Holly, N.C. Nonwoven substrates made from synthetic materials useful in the present invention can also be obtained from a wide variety of commercial sources. Non limiting examples of suitable nonwoven layer materials useful herein include Excell®, Sofspan®, Softex®, Corolind®, and Celestra® polyolefinic nonwovens from BBA Nonwovens, Keyba® 95 IV, a dry formed apertured material, containing about 75% rayon, about 25% acrylic fibers, Duralace® 1236, an apertured, hydroentangled material, containing about 100% rayon, and Duralace® 5904, an apertured, hydroentangled material, containing about 100% polyester all available from PGI/Chicopee, Dayton, N.J.; Sontara® 8877, an apertured hydroentangled material, containing about 50% Nylon and about 50% Pulp available from Dupont Chemical Corp. As previously discussed, substrate materials of the present invention may also include laminates of the above-mentioned materials. Laminates may be combined by any number of bonding methods known to those skilled in the art including, but not limited to, thermal bonding, adhesive bonding including, but not limited to spray adhesives, hot melt adhesives, latex based adhesives and the like, sonic and ultrasonic bonding, and extrusion laminating whereby a polymer is cast directly onto another substrate, and while still in a partially molten state, bonds to one side of the substrate, or by depositing melt blown fiber nonwoven directly onto a substrate. The substrate can be made into a wide variety of shapes and forms including flat pads, thick pads, thin sheets, ball-shaped implements, irregularly shaped implements, and having sizes ranging from a surface area of about 1-1000 cm². The exact size will depend upon the substance stored and released, the desired use, and the overall product characteristics.

Film Substrates

Film substrate materials may be comprised of polyolefin such as polyethylene, including linear low density polyethylene (LLDPE), low density polyethylene (LDPE), ultra low density polyethylene (ULDPE), high density polyethylene (HDPE), or polypropylene and blends thereof with the above and other materials. Examples of other suitable polymeric materials which may be used include, but are not limited to, polyester, polyurethanes, water-soluble polymers, compostable or biodegradable polymers, heat shrink polymers, thermoplastic elastomers, metallocene catalyst-based polymers (e.g., Insite™ available from Dow Chemical and Exxact™ available from Exxon), and breathable polymers, apertured film, macroscopically expanded three-dimensional formed films, foams, filled compositions. As previously discussed, the substrate material may comprise a laminate of one or more elastic layers and one or more non-elastic layers, polymeric films such as thermoplastic films of polyethylene or polypropylene, composite materials such as a film-coated nonwoven material or materials including one or more apertures or apertured regions. Exemplary polyethylene films are manufactured by Clopay Plastic Products Co. of Cincinnati, Ohio under the designation P18-1401 and by Ethyl Corporation, Visqueen Division, of Terre Haute, Ind., under the designation XP-39385. Substrates in this category include membranes, microporous membranes, natural sponges, synthetic sponges, polymeric netted meshes, formed films, and the like. Exemplary water-soluble polymer films include polyvinyl alcohol, polyethylene oxide, copolymer of vinyl alcohol and poly(oxylene)acrylate, and hydroxypropylcellulose.

Substances

Substances used in consumer products such as: shoe polish, paint, lacquer, varnish, metal polish, car wax, window cleaner, floor cleaner, floor polish, plant food serum, cooking oils, fabric cleaners, carpet cleaner, laundry conditioner, laundry detergent, mold release substances, sunscreen, make-up, douche, hemorrhoid cream, beverages, toothpaste, tooth gel, tooth whitening gel, peanut butter, milk, cream, water, hair remover cream, lubricants, spermicides, shaving cream, shaving gel, fragrance, perfume, aftershave substance, antiperspirant, deodorant, pigments, soaps, talc powder, baking powder, flavors, spices, sauces, aqueous rubber latex, oral suspensions, cough medicine, suntan oil, skin tanning colorants, hair dye, hair bleach, tattoo ink, ink, chalk, urine or blood or sweat patient samples for health check or forensic investigations, crayons, printer toner inks, photographic film emulsion, effervescent formulations like Alka-Seltzer®, vitamins, nutritional substances like oils and herbs, fingerprint ink, portable water supply, lubricating oil, skin conditioners, eye shadow, foundation, caulk, rust protector, flushable drain cleaner, eye drops, nose drops, sore throat liquids, mouthwash, skin balm, topical allergy creams, ultra-long lasting chewing gum, kerosene, barbecue charcoal lighting fluid, lighter fluid, chewing tobacco, ketchup and other condiments, soup, space astronaut meals, Jello®, Armor All® tire and vinyl cleaner, paint for curved surfaces like metal pipes, crude oil, pH indicator compositions, pregnancy test formulations, wound ointments, wound hydrocolloid formulations, aloe vera gel for burn sunburn, burn protectant and disinfectant topical formulations, abrasive pastes can be used in the present invention. The substances can comprise a wide range of other optional components. These additional components should be pharmaceutically acceptable when used on mammalian skin. The CTFA Cosmetic Ingredient Handbook, Second Edition, 1992, which is incorporated by reference herein in its entirety, describes a wide variety of non limiting cosmetic and pharmaceutical ingredients. Examples of these and other substances include: abrasives, anticaking agents, emollients, liquid hydrocarbons, petrolatum, phospholipids, polysiloxane polymers, alkyl substituted polysiloxane polymers, antioxidants, vitamins, binders, biological additives, buffering agents, bulking agents, chelating agents, chemical additives, colorants, anti fungal-agents, cosmetic astringents, cosmetic biocides, denaturants, drug astringents, external analgesics, film formers, fragrance components, humectants, opacifying agents, pH adjusters, preservatives, propellants, reducing agents, skin bleaching agents, and sunscreen agents. Other materials include controlled stress dispersions that dry to produce force on the surface applied to—these are used as skin anti-wrinkling agents, or for texture creation on surfaces and so forth. Skin anti-wrinkling agents are formulations including anionic polymers, film forming inorganic colloids, oil soluble organic-inorganic copolymers preferably combined with adhesive and elastic materials for durability, for example, for an all day long skin wrinkle smoothing benefit. Anionic polymers include sodium polystyrene sulfonate such as Flexan® 130 from National Starch corporation, potassium polystyrene sulfonate, polyvinyl sulfate, sodium poly styrene sulfonate—co—maleic anhydride and so on. Film forming inorganic colloids include colloidal silica in water such as Nalco® 1115 from Nalco chemical. Oil soluble organic-inorganic copolymers include MQ® siloxysilicate resin from General Electric. The adhesive and elastic materials include oil soluble elastomers such as styrene-isoprene copolymer such as Kraton™ LV from Shell Oil Company, acrylic elastomer lattices such as Hystretch® styrene-acrylate emulsion from BF Goodrich Company, and so forth.

Physical Properties of Substance

For fluids, the relative surface energy determines the containment and “loadability” and optimum pocket size to use. For flow or release, viscosity is important and is triggered by wiping, humidity, heat, wetting, rubbing, wiping and so on broadens utility to virtually any substance or product composition known in the art from water, glass cleaners, creams, pastes, waxes, film formers, shrinking anti-wrinkle ionic polymers, oil based formulas, greases, pharmaceutical suspensions. For direct transfer, for example in the case of solid substances, release is started by mechanical contact of loaded micropockets with the target surface. Fluid substances employed in the wipes product herein preferably have a viscosity in the range of from about 0.1 centipoise to about 1,000,000 centipoise, the substance viscosity being selected based on the substrate micropocket dimensions and mechanical properties. Preferred viscosity in the case of fluid substances is from 0.5 to 500,000 centipoise. More preferred viscosity is from 1 centipoise to 250,000 centipoise. These viscosities are estimated under the condition of shear at a rate in the range of about 100 s⁻¹. Typical substance viscosities are 1-100 centipoise for cleaning fluids, 500-1000 centipoise for sunscreen lotions, 1000-5000 centipoise for body creams and gels, and 5000-30000 centipoise for surfactant and polymeric pastes. Preferred forms and compositions applicable are listed in the examples section and include all Theological forms in terms of yield point, shear viscosity and creep recovery including but not limited to solid particles, rheopectic shear thickening substances, sticks, gels, pastes, creams, powders, compacts, slurries, mud clay mixtures, concrete mixtures, aqueous dispersions like paints, suspensions, lotions, serums, glues, caked solids, emulsions, microemulsions, nanodispersions, colloids. Note, viscosity modifiers can be included in any of these substances which can change both the viscosity and the rheology profile such as yield point, gel state, extensional viscosity, response of flow to temperature, and so forth.

Optional Components

The substance and the substrate elements of the present invention can be optionally combined with other materials and devices to broaden the use and value of the invention. For example, the substrate-substance combination can be attached to an implement such as a handle, a squeegee, or a brush. Additionally, the substrate-substance combination can be mounted or attached to an energy producing device such as a motor to push or rotate, a vibrating arm to shear, and so on. The substrate-substance combination can also be mounted on manual articles such as gloves, sleeves, and the like. Other materials can also be attached to provide additional features. Non limiting examples of these other materials include: sponges, foams, rigid bracket or holder, handles, straps, and so forth. In addition, materials can be added to package and adapt the product design of the substrate. For example, a peel-off polymer film layer to protect substances contained in the wipe from air or moisture until the point of use.

Preparation of Substrates Combined with Substance

The articles of the present invention are manufactured by adding substances onto or into the substrates. Techniques for coating or impregnating substrates can be accomplished by any means known to those skilled in the art: for example, by spraying, electrostatic spraying, slot coating, printing, splashing, immersing, dipping, soaking, or coating, flood coating, or metered dosing. More specialized techniques, such as Meyer-rod, floating knife or doctor blade, are typically used to impregnate substrates, and may also be used herein. The substrate is then typically packaged in any of the various moisture and vapor impermeable packages known in the art.

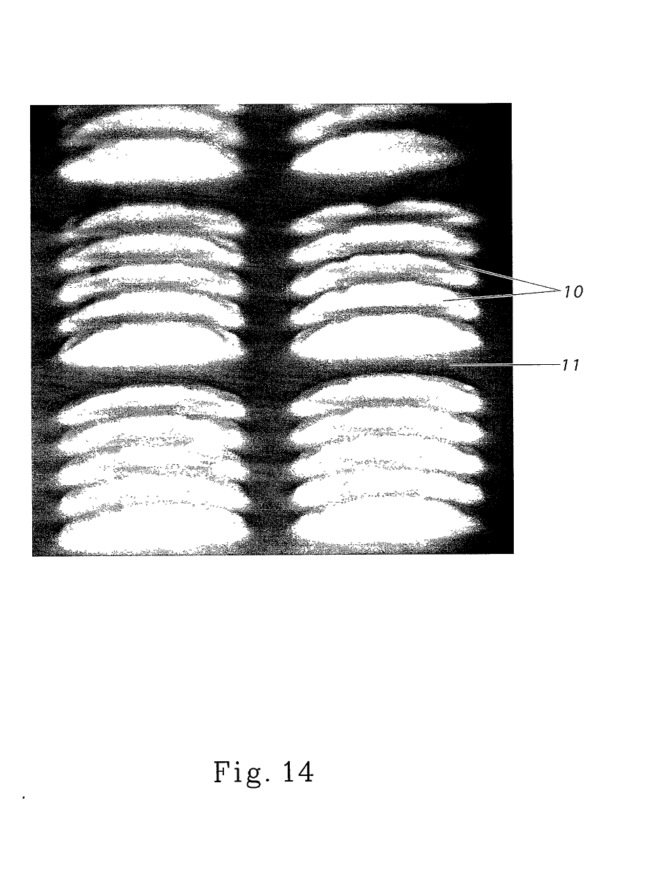

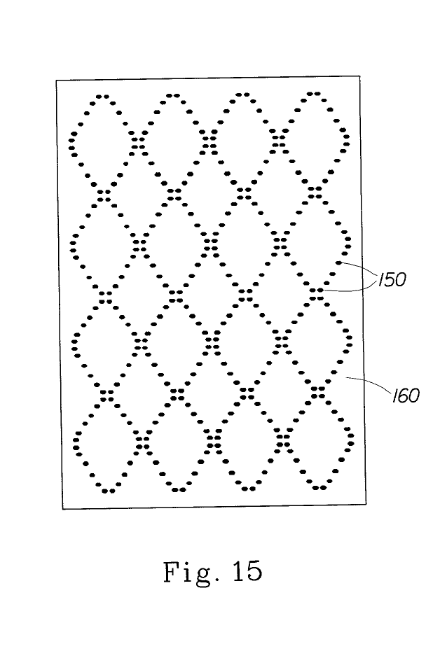

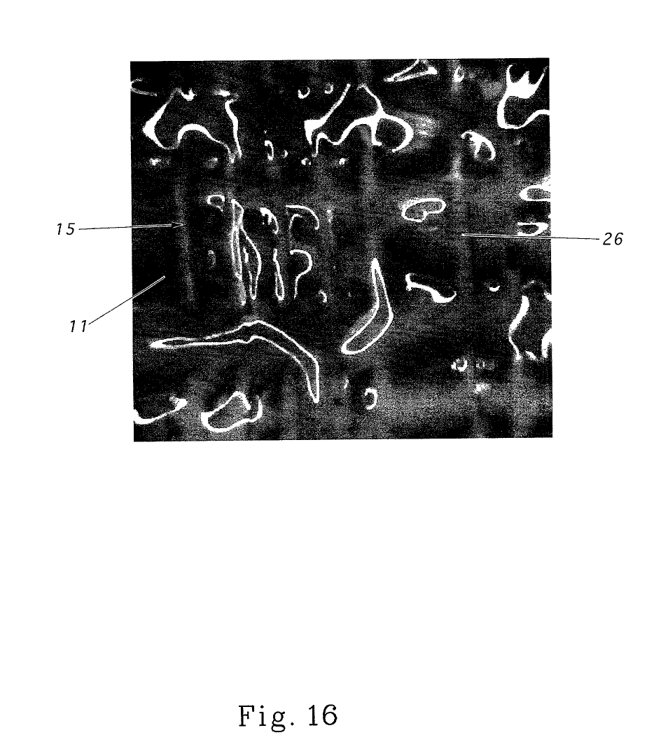

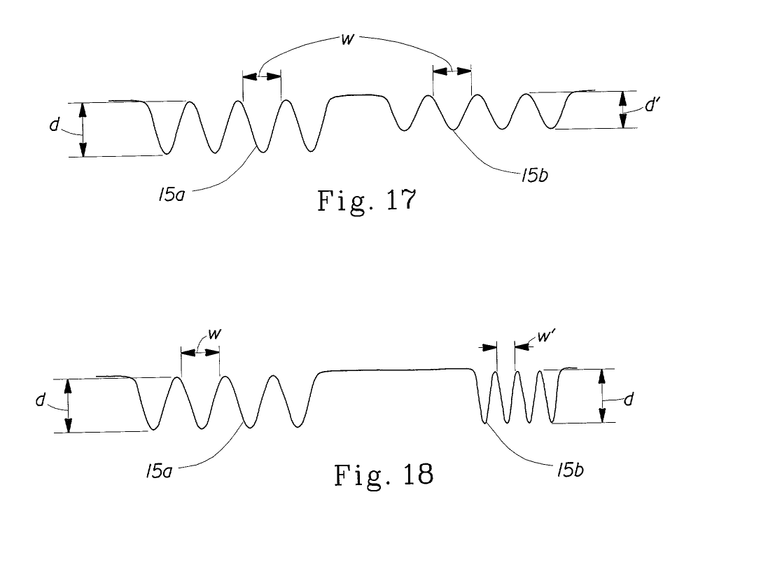

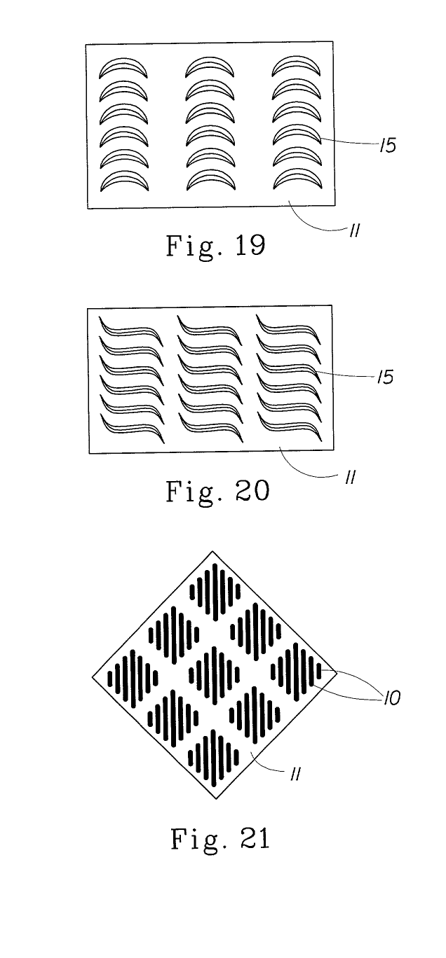

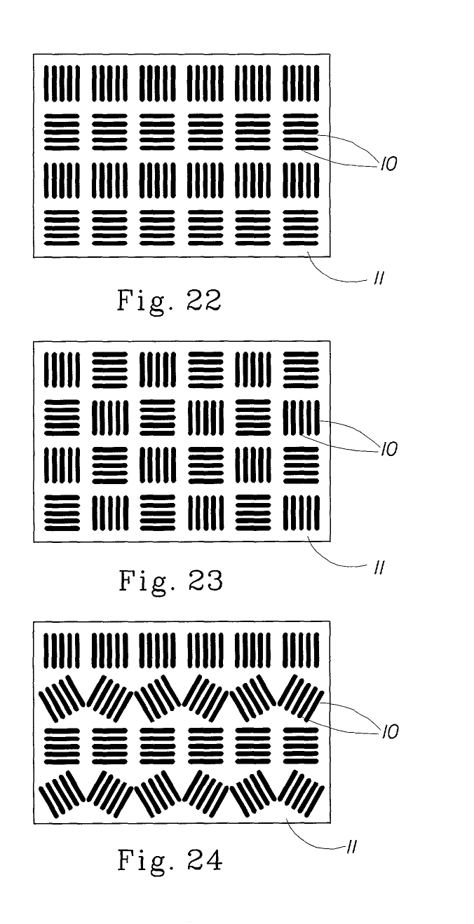

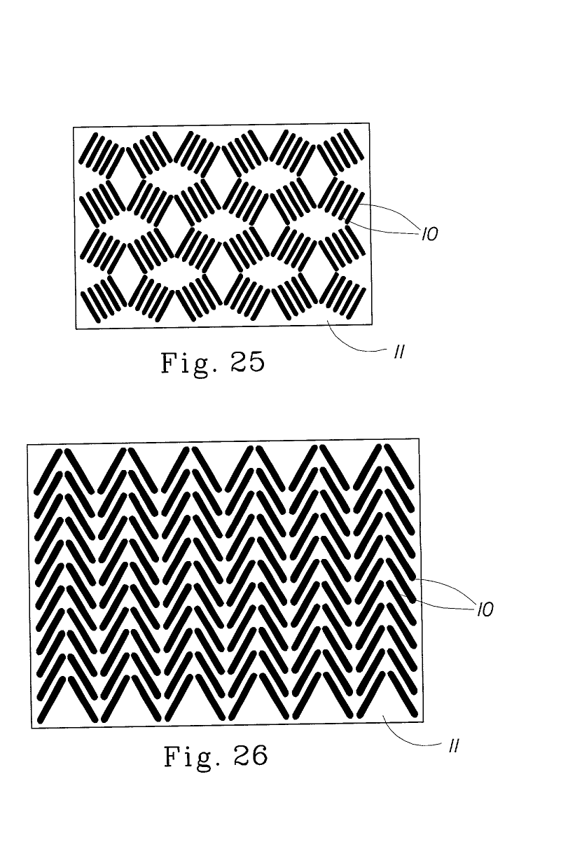

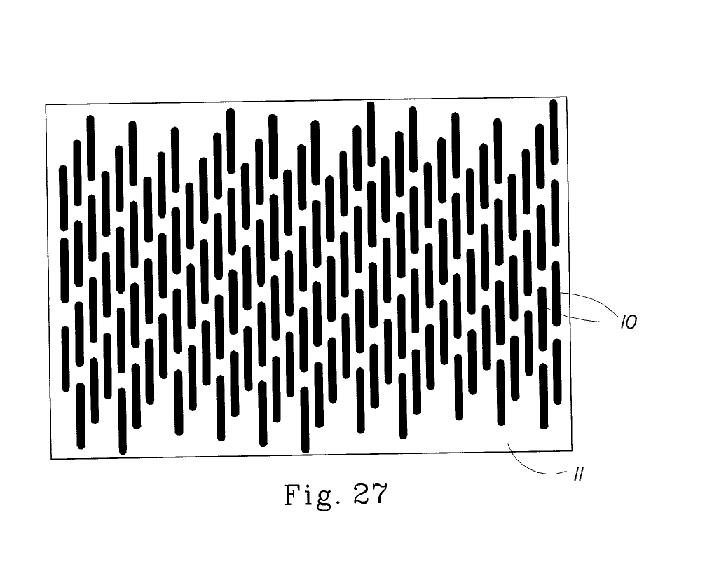

Micropocket making is exemplified in U.S. Pat. No. 5,518,801, incorporated herein, by reference, which describes a process for making substrates with micropockets on the substrates. For example, sheets of nonwoven and film have micropockets formed on them in a hydraulic press using forming plates as exemplified in U.S. Pat. No. 5,518,801. FIG. 14 shows the micropocket pattern 10 on a film 11. The width of a micropocket is about 0.50 mm, the depth of the micropocket is about 0.85 mm, and the length of the micropocket is about 6.0 mm. The micropocket film and nonwovens can be laminated or bonded together using a heat sealer and a bonding plate or an ultrasonic sealer and a bonding plate. A bonding plate 160 having a pattern to bond the substrate layers at multiple points 150 is illustrated in FIG. 15. The substance is then loaded on the substrates, for example by spraying from a pipe drilled with holes and having substance flowing through it. The substance 26 can be loaded through the porous nonwoven side, then reaching and filling the micropockets 15 as illustrated in FIG. 16. About 7.3 ml of the substance is loaded in 300 cm² area of the substrate. This is equivalent to 0.024 milliliter of the substance in one square centimeter of the substrate. This converting step of combining substance with substrate includes but is not limited to immersion, squeegeeing on, spraying, gravure printing, dot matrix deposition, slot coating, printing, filling, dipping, individual ingredient imprinting, impregnating or coating of individual layers followed by bonding of said loaded layers together. This combining of substance with substrate can be done on the whole substrate, individual layers of the substrate, either side of the substrate, deposition in a continuous or discontinuous pattern onto the substrate, and combining of substances into different depths or layers of the substrate. For example, fill substance into one layer of substrate then bond or “cap” off the substrate with a top layer to make a “dry to the touch” article that releases on use. In addition, a wide range of layer bonding patterns and micropocket arrays, geometries, microtextures, depths, widths, and patterns can be used, a few of which are illustrated in FIGS. 17-27.

Method of Using Substances Combined with Substrates

As an example, wipe substrate articles loaded with substance are applied to a surface such as skin, flooring, and the like to deliver substance to perform the desired function. The substrates can be used by holding in the hand of consumer and stroking across a surface or attaching substrate plus substance to an implement such as broomstick, gripping device such as tongs or tweezers, moving plate or machine, or attaching to a surface that subsequently moves to induce release for example a wrap on a consumer's arm. Preferred methods of use of the present invention is in the context of products that undergo rubbing or shearing, especially to redirect, store, temporarily immobilize, release, and/or deliver substances. This includes but is not limited to hygiene articles, cleaning cloths, feminine hygiene garments, catamenial pads, incontinence garments, liners, and bandages.

Additional examples of uses of the present invention include substance loaded packaging liners or lids; shower caps and other personal hygiene articles in contact with the body such that the contained substance can be transferred; condoms with lubricant or spermicide to be released upon shearing action during intercourse; filters with substances that assist filtration and air or fluid cleaning; foundation article that delivers all forms of color and foundation to skin during spreading or dabbing including delivery of discrete individual ingredients like pigments and glycerin that are separately loaded into different individual micropockets or at different layers of micropocket substrate; shoe cream wipe, leather conditioning wipe; car window cleaning wipe, wet/dry wipe substrate or sleeve with hand insert for hard surface cleaning; wettable soap matrix facial washcloth; body lotion or body wash pad; freshen-up wipe; deodorant delivering wipe; pantyliner with douche; pantyhose with lotion release during shear of micropockets with leg motion and contact; panty pad which releases anti-fungal medicine during moving and sitting; oil or butter wipe for no-mess application, for example, coating a meat before roasting and for efficient low fat cooking; foot insole with shear-triggered air freshener and odor remover; child's shampoo wipe with low water use and less soap in the eyes; hair colorant and bleach placed into discrete layers of micropockets or in different individual micropockets to prevent interaction in pack, then combine on the hair in use; artificial skin suntan color toning wipe to tan whole body; massage oil in micropocket substrate that keeps releasing and no mess on massager's hands; hair removal wipe like need to protect hand from chemicals and improve contact with skin for hair removal; transdermal analgesic in a wipe form or leave on skin form such that release increases with relative motion of skin and micropocket substrate; pet bath or medication applicator for wash on the go and with minimal water that dogs and cats hate; glass cleaner in a disposable wipe or fill micropockets and re-use; surface cleaning wipe with micropocket film on one side and paper towel on the back to both release and remove; paint wipe without needing brush and no mess on hands plus disposable and keep paint fresh; car wax liquid wipe no hand mess and easy to cover large areas; abrasive cloth containing fluid suspension of abrasives that release during rubbing and shearing; tooth paste strip which fits over finger like a cot and releases paste on rubbing across teeth for portable and disposable dental hygiene or for teaching children how to clean teeth prior to their ability to manually handle a toothbrush; clothing insert pad with anti-irritant or deodorant or therapeutic like psoriasis medication so that when clothes rub, medicine is delivered to chapped or ill skin areas; hemorrhoid under pant substrate to release Preparation H® or cool and soothe with aloe vera gel slowly over time and during shearing natural motions or shifting while seated on a chair over long time periods; child knee pads with lotion substance to protect knees during crawling; hand lotion wipe for winter chapped hands; deliver to skin in spots or all over skin anti-aging additives; anti-cellulite medicine wipe; anti-wrinkling wipe loaded with controlled stress polymers; heel protecting lotion for thick hard skin to soften over day; genital cleansing cloth: medical wipe containing disinfectant; wound bandage with micropockets that releases gel or lotion during rubbing or painful motions of skin relative to bandage; liner for fractured bone cast the releases substance and pain reliever gel when moved to counteract; Orajel® benzocaine insert into chewable fabric or rub onto painful teeth and gums micropocket cloth-plastic strip; discs with micropockets loaded with floor wax for machine application to wood or composite floors; non-drip but high liquid content anal wipes with micropockets for high soil events occurring far from running water source; facial cleansing cloth with lipid, low viscosity conditioners in micropockets, surfactant on nonwoven layer; nose handkerchiefs with antihistamine suspensions or cold remedies to deliver protective nasal materials to external and internal nose skin surface while sneezing or wiping runny nose.

Another example of using shear responsive micropocket substrate is delivering film forming substances including, but not limited to paint, latex, adhesives, external analgesics, cosmetics, anti-wrinkle materials, and high polymer containing makeup. The film forming substances can be delivered in the form of a substantially discontinuous film or patterned coating on a target surface for increased durability and fracture resistance. When the substance used is cosmetics or an anti-wrinkle agent, substantially discontinuous or discrete patterns of films on skin can provide extra comfort in addition to increased durability and fracture resistance of the makeup film. The shear responsive micropocket substrate can also be used as a mold to create a replica of itself, which is also shear responsive, from the substances contained in it. The shear responsive substrate can be loaded with film forming substances, as mentioned above, which can be delivered onto a target surface as a continuous or discontinuous shear responsive film having the same micropocket structure as that of the substrate containing these substances. Examples of such application include but are not limited to delivering flexible and shear responsive adhesive coatings on a surface or between two joined surfaces, comfortable high-polymer content makeup film for skin, medication applied as a comfortable film externally on moving joints, such as hip, knee, ankle, elbow, shoulder, wrist, finger-joints, and neck of human or animal body.

### EXAMPLES

The following examples further illustrate preferred embodiments within the scope of the present invention. The examples are given solely for the purposes of illustration and are not limitations of the present invention as many variations of the invention are possible without departing from its spirit and scope. The examples illustrate one or more of the advantages of the present invention, that is, high efficiency of release, sustained release, substantial shear responsiveness, dual sidedness, high substances storage in a thin substrate, ability to load different rheology materials into a wipe article. In the examples, as illustrated in FIGS. 1-4, micropockets are described with length L between about 0.1 mm and about 100 mm, more preferably between about 0.5 mm and about 50 mm; width W between about 0.1 mm and about 10 mm, more preferably between about 0.2 mm and about 5 mm; and depth D of the micropockets is between about 0.05 mm and about 10 mm, more preferably between about 0.1 mm and about 5 mm. Correspondingly, each micropocket holds between about 100 picoliter to about 10 milliliter, more preferably between about 0.01 microliter to about 1 milliliter. The number of micropockets in example embodiments is described with micropockets per unit area from about 1 micropocket per cm² to about 100 micropockets per cm², more preferably from about 2 micropockets per cm² to about 50 micropockets per cm². Examples 1-2 have preferred micropockets per unit area of 6 micropockets per cm² with a preferred length of 10 mm, a preferred width of 0.5 mm, and a preferred depth of 0.9 mm. Examples 3-6 have preferred micropockets per unit area of 15 micropockets per cm² to 20 micropockets per cm² with a preferred length between 1 mm to 5 mm, a preferred width between 0.4 mm to 0.5 mm, and a preferred depth between 0.4 mm to 1.1 mm. The volume of substance loaded onto the substrate articles in the examples below is typically between 1 milliliter to 50 milliliter for a typical 10 cm by 10 cm sized wipe article.

### Example 1

### Foundation Applicator, Hand and Body Lotion Pad

Substrate: A laminate is provided comprising a linear low density polyethylene film with micropockets, base film approximately 30 micron thick, designated as “1401” available from Clopay Plastic Products Co., Cincinnati, Ohio; a vacuum apertured polyethylene film, like “VisPore™” film available from Tredegar Film Products, Richmond, Va.; and a polyethylene nonwoven 30 grams per square meter basis weight, designated as “Corolind®” available from BBA Nonwovens Germany GmbH, Piene, Germany. Substrate components are cut into circular shapes 80 millimeters in diameter then product is prepared as described below. Note, a batting layer can also optionally be included at the back of the micropocket film to add grip and thickness and create pad-like form. This batting can comprise any thick, relatively light material including bubble wrap; felt; air laid cotton or cellulose; and/or styrofoam.

Substance: Foundations and skin lotions or gels in the form of a compact, oil in water emulsion, water in oil emulsion, suspension containing powders, optionally with fluids such as oils, silicones, water, alcohol can all be used. Typical substance loading is between 5 milliliter and 20 milliliter in this pad example for a pad approximately 10 cm by 10 cm in area.

Preparation: Substance is spread or sprayed onto the low density polyethylene film with micropockets. After filling excess foundation is removed, the loaded film is laminated to the apertured film and nonwoven by ultrasonic bonding, for example. Alternatively, the entire laminated substrate is filled by squeegeeing the foundation through the porous side of the substrate.

### Example 2

### Sunscreen Wipe, Baby Wipe, Antiperspirant/Deodorant/Refreshment Wipe

Substrate: A laminate is provided comprising two layers of polypropylene nonwoven 33 grams per square meter basis weight, designated as “Sofspan®” available from BBA Nonwovens, Simpsonville, S.C., each with micropockets. Alternatively, a three layer wipe with a Clopay 1401 micropocket film laminated between two layers of “Sofspan®” nonwoven. Substance: Sunscreen, baby, antiperspirant and other lotion formulations are provided ranging from powdery combinations of actives such as zinc oxide, titanium dioxide, and octyl methoxycinnamate to formulations of these and cleansing, fragrance, water and other ingredients in the form of creams, lotions, foams, mousses, suspensions and liquids.

Preparation: The laminate layers are bonded with a pattern bonding to prevent sliding and shifting of layers during use. Substrate is filled with substance by spraying then squeegeeing the sunscreen lotion, for example onto the substrate. This can be done on individual wipe articles, or on a line followed by cutting, folding, and packing steps. Typical substance loading is between about 2.5 milliliter to about 5 milliliter in a wipe approximately 10 cm by 10 cm in area.

### Example 3

### Wet Cleaning Cloth for Hard Surfaces

Substrate: The substrate comprises a 66 grams per square meter hydroentangled polyester nonwoven with polypropylene scrim, designated “IDDU Apex®” substrate from PGI Nonwovens, Rogers, Ark., laminated to a linear low density polyethylene film with micropockets, base film designated as “1401” available from Clopay Plastic Products Co., Cincinnati, Ohio; and two layers of polypropylene nonwoven of 15 grams per square meter basis weight each, designated as “Sofspan®” available from BBA Nonwovens, Simpsonville, S.C. Optionally, a layer of “Bounty®” paper towel, manufactured by The Procter and Gamble Company, Cincinnati, Ohio, is included. Optionally, the substrate is clipped or wrapped onto the head of an implement with a long handle. Optionally, a micropocket film and/or micropocket nonwoven extrusion laminated or coated with elastomeric scrim grabber material is also used.

Substance: Cleanser formulations are used comprising water, volatile solvents such as propylene glycol, alcohol, surfactants, and fragrance. These are typically low viscosity liquids.

Preparation: Laminate all layers of substrate using ultrasonic or thermal bonding. Squeegee or spray substances, fold, and package. Typical substance loading is between 2 milliliter to 50 milliliter in a cloth approximately 15 cm by 30 cm in area.

### Example 4

### Facial Wash and Conditioning Cloth

Substrate: A laminate is used comprising a micropocketed 50/50 bicomponent polyethylene-polypropylene sheath-core nonwoven 60 grams per square meter basis weight, base nonwoven designated as “Softex®” available from BBA Nonwovens, Simpsonville, S.C.; and a linear low density polyethylene micropocket film, where base film is 30 micron thick, designated as “1401” available from Clopay Plastic Products Co., Cincinnati, Ohio.

Substance: The substance used can comprise skin conditioners such as glycerin petrolatum and dimethicone silicone fluid and solid/paste/liquid cleansing formulation of surfactants.

Preparation: Deposit by slot coating the surfactant solid or paste onto the micropocket nonwoven. Spray or spread conditioner fluid onto micropocket film. Bond the two layers together with ultrasonic or thermal bonding. Optionally, make the nonwoven cleansing layer peelable by edge bonding with a pressure-sensitive adhesive or insert a optional water soluble polymer sheet layer such as polyvinylalchol film in between the nonwoven and film layers. Typical substance loading is between 0.1 milliliter to 5 milliliter of each individual substance in a wipe approximately 10 cm by 10 cm in area.

### Example 5

### Two Sided Cleaning Sleeve

Substrate: A three-layer laminate is used comprising as a first layer, a 66 grams per square meter hydroentangled polyester nonwoven with polypropylene scrim, designated “IDDU Apex®” substrate from PGI Nonwovens, Rogers, Ark.; a second layer, laminated to the first layer, comprising a linear low density polyethylene film micropockets, where base film is 30 micron thick, designated as “1401” available from Clopay Plastic Products Co., Cincinnati, Ohio; and a third layer, bonded to first two layers along the two opposite edges, comprising a paper towel, like “Bounty®” Paper towel, manufactured by The Procter and Gamble Company, Cincinnati, Ohio.

Substances: Cleanser formulations are used comprising water, volatile solvents such as propylene glycol, alcohol, surfactants, and optionally, fragrance. These are typically low viscosity liquids.

Preparation: Laminate first and second substrate layers by ultrasonic bonding. Bond two opposite edges of third layer to the laminate to make a sleeve using ultrasonic or thermal bonding. Squeegee or spray substance on the nonwoven side of the laminate, folds, and package. Typical substance loading is between 1 milliliter to 15 milliliter of each individual substance in a wipe approximately 10 cm by 10 cm in area.

### Example 6

### Garment Wipe for Stain Treatment or as Pretreatment Prior to Dry Clean

Substrate: A three-layer laminate is used, about two-fingers wide and long, with as a first layer, a 30 grams per square meter polypropylene nonwoven, like “Sofspan®” from BBA Nonwovens, Simpsonville, S.C.; second layer, laminated to the first layer, comprising a linear low density polyethylene film micropockets, where the base film is 30 micron thick, designated as “1401” available from Clopay Plastic Products Co., Cincinnati, Ohio; and a third layer, bonded to first two layers along the two opposite edges, comprising a paper towel, like “Bounty” Paper towel, manufactured by The Procter and Gamble Company, Cincinnati, Ohio.

Substance: The substance used can be cyclomethicone for stain treatment of leather articles, or “Dryel®” pre-treatment from The Procter and Gamble Company, Cincinnati, Ohio, for garment stain pre-treatment.

Preparation: Laminate first and second substrate layers by ultrasonic bonding. Bond two opposite edges of third layer to the laminate to make a sleeve using ultrasonic or thermal bonding. Squeegee or spray substance on the nonwoven side of the laminate, fold, and package. Typical substance loading is between 0.1 milliliter to 10 milliliter in a laminate size 6 cm×4 cm.

What is claimed is:

1. A wipe article comprising:
a) A substrate, said substrate having a plurality of micropockets, said micropockets having a length, a width, and a depth, wherein the ratio of said depth to said width is at least 1:2; and
b) at least one substance in said micropockets, said substance remaining in said micropockets until said substrate is subjected to a shear force while in contact with a target surface, said shear force causing walls of said micropockets to substantially deform, and release said substance to said target surface.

2. The substrate of claim 1, wherein ratio of said depth to said width of said micropockets is between about 0.7:1 and about 2.8:1.

3. The substrate of claim 1, wherein:
a) said length of said micropockets is between about 0.1 mm and about 100 mm;
b) said width of said micropockets is between about 0.1 mm and about 10 mm; and
c) said depth of said micropockets is between about 0.05 mm and about 10 mm.

4. The substrate of claim 1, wherein said substrate has from about 1 micropocket per cm² to about 100 micropockets per cm².

5. The substrate of claim 1, wherein said substrate is a film substantially impervious to at least one substance contained in said micropockets of said substrate.

6. The substrate of claim 1, wherein said substrate is a porous film substantially pervious to at least one said substance contained in said micropockets of said substrate.

7. The substrate of claim 1, wherein said substrate is a nonwoven.

8. The substrate of claim 1, wherein said substrate is elastomeric.

9. The substrate of claim 1, wherein said substrate has at least two layers, wherein at least one of said layers has a plurality of said micropockets.

10. The substrate of claim 9, wherein at least one said layer of said substrate is a film substantially impervious to said substance contained in said micropockets.

11. The substrate of claim 9, wherein at least one said layer of said substrate is a porous film substantially pervious to at least one said substance.

12. The substrate of claim 9, wherein at least one said layer of said substrate is a nonwoven.

13. The substrate of claim 9, wherein at least one said layer of said substrate is elastomeric.

14. The film of claim 5, wherein said film is comprised of a polymer selected from the group consisting of polyethylene, polypropylene, polyvinylalchol, polyethylene-polypropylene copolymers, and mixtures thereof.

15. The porous film of claim 6, wherein said film is a polymer selected from the group consisting of polyethylene, polypropylene, polyvinylalchol, polyethylene-polypropylene copolymers, and mixtures thereof.

16. The substance of claim 1, wherein said substance is selected from the group consisting of cleansing agents, skin care agents, medicinal agents, suncare agents, analgesic agents, emollients, lubricants, colorants, preservatives, condiments, deodorants, antiperspirants, fragrances, adhesives, cooking oil for basting, conditioning agents, humectants, shoe care agents, and insect repellants, and mixtures thereof.

17. The substance of claim 1, where in said substance is a skin anti-wrinkle agent.

18. The substance of claim 16, wherein said substance is in a form selected from the group of a rigid gel, cream, substance, oil in water emulsion, water in oil emulsion, tonic, suspension, dispersion, wettable and redispersible solid.

19. The substrate of claim 9, wherein said substrate is to be used as a facial wash cloth, wherein said substrate has at least two layers, the first layer of said substrate contains a cleansing substance, the second layer of said substrate contains a conditioning substance to be released after said cleansing substance in said first layer is released.

20. A method of applying at least one substance to a target surface, said method comprising:
a) providing a substrate having a plurality of micropockets containing said substance, said micropockets having a length, a width and a depth, wherein the ratio of said depth to said width is at least 1:2;
b) placing said substrate in contact with said target surface; and
c) applying shear forces along the surface of said substrate while said substrate is in contact with said target surface, said shear forces causing walls of said micropockets to substantially deform, and release said substance, to said target surface.

21. The method of claim 20 wherein said substrate is a clothing insert, a shoe insole, or a panty liner, said substance is selected from the group consisting of odor reducing agents, cleaning agents, anti-fungal medications, and mixtures thereof, and wherein said substance is released during motion of a clothing wearer.

22. A method of delivering a shear responsive film onto a target surface, said method comprising:
a) providing a substrate having a plurality of micropockets containing a film forming material, said micropockets having a length, a width and a depth, wherein the ratio of said depth to said width is at least 1:2;
b) placing said substrate in contact with said target surface; and
c) applying shear forces along the surface of said substrate, said shear forces causing the walls of said micropockets to substantially deform and transfer said film forming material to said target surface in the form of a replica of said substrate.

23. The wipe article of claim 1, wherein said wipe article is a sunscreen wipe, a skin lotion pad, a floor cleaning cloth, an antiperspirant or refreshment wipe, or a baby wipe.
